(19) United States
(12) Patent Application Publication
(10) Pub. No.: US 2004/0077831 A1
(43) Pub. Date: Apr. 22, 2004

(54) Method for purifying a biological composition

(76) Inventors: John Chapman, Newton, MA (US); Andrei Purmal, Waltham, MA (US); James Hope, Newtonville, MA (US)

Correspondence Address: Ivor R. Elrifi, Esquire, MINTZ, LEVIN, COHN, FERRIS,, GLOVSKY and POPEO, P.C., One Financial Center, Boston, MA, 02111, US

(21) Appl. No.: 10/055,143

(22) Filed: Jan. 22, 2002

Related U.S. Application Data
(63) Continuation-in-part of application No. 09/945,979, filed on Sep. 4, 2001.
Application No. 09/945,979 is a continuation-in-part of application No. 09/827,491, filed on Apr. 6, 2001, now abandoned.
(60) Provisional application No. 60/263,417, filed on Jan. 22, 2001.

(51) Int. Cl. A01N 1/02; C07K 14/47

(52) U.S. Cl. 530/350; 435/2

(57) ABSTRACT

Disclosed is a method for removing an analyte from blood cells that results in a preparation of blood cells in which the level of the residual analyte is significantly reduced in the cell population. The method can be performed on large volume blood cell suspensions, and the cells prepared in this manner remain viable following prolonged storage and are suitable for therapeutic use, e.g. in transfusion applications. A preferred blood cell preparation is one that includes a red blood cell (RBC) population.

### RELATED APPLICATIONS

This application claims priority to U.S. Ser. No. 09/945,979, filed Sep. 4, 2001; U.S. Ser. No. 09/827,491, filed Apr. 6, 2001; and to U.S. S No. 60/263,417, filed Jan. 22, 2001. The contents of these applications are incorporated herein by reference in their entireties.

### FIELD OF THE INVENTION

The invention relates to methods for removing analytes, such as prion proteins, from biological compositions, such as blood.

### BACKGROUND OF THE INVENTION

Cellular blood products (such as red blood cells (“RBCs”) and platelets) may be subjected to extensive purification and storage procedures prior to being transfused into a patient. Purification procedures can include inactivation and/or removal of contaminating pathogens (e.g., viruses, bacteria, protozoa) and removal of undesired proteins and nucleic acids. It is recognized that purification of red cells can affect the shelf life of the stored blood products and can also affect the survival of the blood cells in the body upon transfusion.

During storage, blood compositions, such as red blood cells, undergo morphological and biochemical changes, and can lyse, which is termed hemolysis. Morphological and biochemical changes can affect the fluidity of the cell membrane of red cells and also the ability of the hemoglobin in these cells to deliver oxygen to the tissues. Morphological changes that occur during storage ultimately lead to the development of spicules on the cells (echinocytosis). These spicules can bud off as vesicles, radically changing the surface-to-volume ratio of the cells and their ability to deform on passing through narrow channels. Such abnormal and damaged cells are typically removed from the blood stream. Accordingly, cells are considered suitable for transfusion only if a minimal number of cells (typically at least 75% of the red cells) are circulating 24 hours following the transfusion.

In certain circumstances it can be desirable to extend the time for which blood cells can be stored. For example, autologous blood products, i.e., blood products removed from a donor prior to a surgical procedure and re-introduced into the donor during or after surgery, may expire before the surgery can be performed. It has also been proposed that blood products be stored for several months to allow retesting the donor for evidence of diseases caused by infectious agents which do not manifest themselves until several weeks after infecting a donor. These diseases can include, e.g. AIDS or hepatitis diseases.

### SUMMARY OF THE INVENTION

The invention is based in part on the discovery of a method for removing an analyte from blood cells that results in a preparation of blood cells in which the level of the residual analyte is significantly reduced in the cell population. The method can be performed on large volume blood cell suspensions, and the cells prepared in this manner remain viable following prolonged storage and are suitable for therapeutic use, e.g. in transfusion applications. A preferred blood cell preparation is one that includes a red blood cell (RBC) population.

In one aspect the invention provides a method for reducing the amount of extracellular fluid, e.g. plasma, in a blood cell suspension. The method includes providing a large volume blood cell suspension that includes blood cells and extracellular fluid, and washing the blood cell suspension with a wash solution under conditions sufficient to lower the amount of the extracellular fluid in the blood cell suspension at least 10³-fold relative to the amount of extracellular fluid in the starting suspension. Also provided are blood cell compositions produced by the washing methods of the invention. Also provided is a method of transfusing the blood cell composition produced by the washing methods of the invention to a recipient.

In preferred embodiments, the blood cell suspension to be washed is provided in a volume that is greater than 40 mL, 50 mL, 75 mL, 100 mL, 200 mL, 300 ml, 400 ml or even 1 L.

In preferred embodiments, the washed blood cells retain viability after prolonged storage at 4° C. in an appropriate storage solution. For example, in preferred embodiments, the washed blood cells retain viability after 24 days of storage at 1-6° C., preferably 4° C. In preferred embodiments, the washed blood cells retain viability after 24 hours, 2 days, 7 days, 14 days, 21 days 28 days, 35 days, 40 days, 42 days or more of storage at 1-6° C. preferably at 4° C.

The washing method of the invention can be used to significantly reduce the concentration of any residual analyte in a blood cell suspension relative to the concentration of the analyte in the starting blood cell suspension. In some embodiments, the concentration of more than one analyte may be reduced. In some embodiments, the analyte is associated with the plasma membrane of a cell, e.g., on the extracellular surface of the plasma membrane. In some embodiments, the analyte is present in extracellular fluid of a blood cell suspension.

In some embodiments, the analyte is a small molecule. In a preferred embodiment of the invention a method is provided for substantially reducing the concentration of an undesired small molecule in a donor blood cell suspension which may be potentially harmful to a recipient such as a drug, an anti-pathogenic agent or a cell preserving agent. As used herein, a “small molecule” is a molecule having a mass of less than about 1000 daltons. Examples of a small molecule capable of removal by the methods of the invention are glycerol, dimethyl sulfoxide (DMSO), ethyleneimine oligomers and derivatives thereof, phenothiazine derivatives, psoralens, acridine derivatives, riboflavin or drugs, such as anticoagulants or antibiotics. In some embodiments where the analyte is a small molecule, and the washing method of the invention reduces the concentration of any residual analyte by a factor of at least 100, preferably at least 1000 fold relative to the concentration of the analyte in the starting blood cell suspension. In a preferred embodiment, the method of the invention, therefore, substantially reduces the concentration of an undesired small molecule in a donor blood cell suspension, thereby reducing undesired or the risk of undesired pharmacologic, immunologic, or toxicologic affects in a recipient while maintaining the therapeutic suitability of the blood cell suspension, even after prolonged storage prior to transfusion (greater than 3 days for platelets, and greater than 14 days for red blood cells).

In other embodiments, the analyte is a molecule larger than 1000 Dalton. For example, the analyte can be a macromolecule such as a nucleic acid or protein. In a preferred embodiment of the invention a method is provided for substantially reducing the concentration of an undesired macromolecule in a donor blood cell suspension which may be potentially harmful to a recipient such as: prion proteins which can cause neurologic disorders; enzymes, antibodies and cytokines that can produce inflammatory and febrile reactions in a recipient; or plasma proteins that can cause allergic reactions, Examples of protein analytes whose levels in blood cell suspension are reduced according to the methods of the inventions are prion proteins, cytokines (e.g., interleukin 1 beta tumor necrosis factor alpha interleukin 6, interleukin 8, interleukin 10), inflammatory enzymes (neutrophil elastase, cathepsins, serine proteinases), anaphylatoxins (e.g., C3a, C5a, bradykinin); immunoglobulins (e.g., IgG, IgM, and IgA). In a preferred embodiment, the method of the invention, therefore, substantially reduces the concentration of an undesired macromolecule in a donor blood cell suspension, thereby reducing undesired or the risk of undesired reactions in a recipient while maintaining the therapeutic suitability of the blood cell suspension, even after prolonged storage.

In other embodiments, the analyte to be removed and/or reduced is a cell, e.g. bacteria, protozoa, or a virus particle, particularly an extracellular virus particle. In particularly preferred embodiments, the cell to be removed and/or reduced by the methods of the invention is a leukocyte (including leukocyte membrane fragments). In a preferred embodiment, the method of the invention, therefore, substantially reduces the concentration of an undesired cell, cell fragment in a donor blood cell suspension, thereby reducing undesired reactions in a recipient while maintaining the therapeutic suitability of the blood cell suspension, even after prolonged storage.

Accordingly, a method of the invention is provided for reducing the occurrence of or reducing the risk of undesired reactions in a recipient by reducing the concentration of a potentially harmful analyte in a blood cell suspension prepared from donor blood. In a preferred embodiment, the method includes reducing the concentration of an undesired analyte at least 10 fold, 100 fold, 10³-fold, 10-fold, 10⁵-fold, or 10⁶-fold relative to the starting concentration of the blood cell suspension. The undesired analyte can be a small molecule or a large molecule. By “small molecule” is meant a molecule with a molecular weight of less than 1000 Daltons. Examples of the foregoing may include glycerol, DMSO, ethyleneimine oligomer, psoralens, phenothiazine-based agents, acridine-based agents, riboflavin or a drug which may include any drug which is recognized by American Association of Blood Banks or the FDA or the U.S. military as being a disqualification for donating.

The analyte can alternatively, or in addition, be a molecule larger than 1000 Daltons. For example, the analyte can be a macromolecule such as a nucleic acid or protein. Examples of protein analytes whose levels in blood cell suspension are reduced according to the methods of the inventions are prion proteins, particularly pathogenic prion protein. Other examples of analytes that are removed by the methods of the invention can include, cells, e.g. leukocytes, microbial pathogens (such as bacteria, fungal or protozoan organism), or infectious viral agents.

In preferred embodiments, washing includes centrifuging the blood cell suspension to form a packed cell fraction and a supernatant comprising the extracellular fluid, removing the supernatant from the packed cell fraction, adding washing solution to the packed cell fraction, and resuspending the packed cell fraction in the washing solution to form a resuspended cell suspension. If desired, the centrifugation and resuspending steps can be repeated, e.g., for three, four, five, six, seven, eight, nine, ten, eleven, twelve, thirteen, fourteen, fifteen, sixteen, seventeen, eighteen or more times. The number of times the centrifugation and resuspension steps are repeated will depend on the desired fold reduction of the extracellular fluid and/or analyte and the ratio of wash solution to extracellular fluid used in each wash step. The greater the ratio of wash solution to extracellular fluid the fewer the number of wash cycles that will be required to achieve the desired fold reduction of the extracellular fluid. For example, a 300 ml red cell concentrate with a 50% hematocrit contains 150 ml of RBCs and 150 ml of extra-cellular fluid. If the blood is centrifuged to achieve a red cell pellet of 80% hematocrit and the remaining extra-cellular fluid is removed, the residual extra-cellular fluid in the RBC concentrated is 30 ml. If the blood is resuspended by the addition of 270 ml then a dilution of 1/10 has been achieved. This process can be repeated over several cycles. The first, second, and third cycles achieve extra-cellular dilution effects of 10-fold, 100-fold and 1,000-fold, respectively.

The starting or the resulting blood cell suspension washed by the methods of the invention may be assayed, by methods known in the art, e.g., for desired fold reduction of analyte by detection methods specific for the analyte and/or for viability.

In some embodiments, the wash solution of the invention is phosphate buffered saline. In some embodiments, the wash solution of the invention comprises 50 mM or less phosphate, e.g., about 12 to 30 mM phosphate. This embodiment of the invention is based in part on the discovery that washing a blood cell suspension with a phosphate buffered wash solution results in an increase in the reduction of an analyte (e.g. an ethyleneimine oligomer or derivative) compared to a cell suspension washed with a solution that does not comprise phosphate. Moreover, the cell suspension of the invention washed with a phosphate buffered solution comprises a lower hemolysis level and maintains a higher ATP concentration compared to a cell suspension washed in an unbuffered saline solution.

In preferred embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells. In particularly preferred embodiments, the methods of the invention can be used to remove and/or reduce extracellular fluid or extracellular fluid and an analyte in blood cell suspensions that include mammalian (such as human) red blood cell concentrates or platelet concentrates or leukocyte concentrates. In preferred embodiments, the blood cell suspension includes mammalian a-nucleated cell concentrates. In a particularly preferred embodiment, methods of the invention can be used to remove and/or reduce extracellular fluid or extracellular fluid and an analyte in a mammalian (such as human) red blood cell concentrate.

In preferred embodiments, washing lowers the amount of residual extracellular fluid at least 10⁴-fold, 10⁵-fold or 10⁶-fold compared to the amount present in the starting cell suspension. In more preferred embodiments, washing lowers the amount of the residual extracellular fluid at least 10⁷-fold, 10⁸-fold, 10⁹-fold, 10¹⁰-fold or 10¹¹-fold compared to the amount present in the starting cell suspension. In even more preferred embodiments washing lowers the amount of residual extracellular fluid at least, 10-fold, 10¹³-fold, 10¹⁴-fold, 10¹⁵-fold, 10¹⁶-fold, 10¹⁷-fold, or 10 ¹⁸-fold compared to the amount present in the starting cell suspension.

In preferred embodiments, washing lowers the concentration of an analyte at least 10²-fold, 10³-fold or 10⁴-fold in the cell suspension compared to the concentration in the starting blood cell suspension. In preferred embodiments, washing lowers the concentration of the analyte at least 10⁵-fold or 10⁶-fold in the cell suspension compared to the concentration in the starting blood cell suspension. In further preferred embodiments, washing lowers the concentration of the analyte at least 10⁷-fold, 10⁸-fold, 10⁹-fold, 10¹⁰-fold or 10¹¹-fold in the cell suspension compared to the concentration in the starting blood cell suspension. In additionally preferred embodiments, washing lowers the concentration of the analyte at least 10¹²-fold, 10¹³-fold, 10¹⁴-fold, 10¹⁵-fold, 10¹⁶-fold, 10 ¹⁷-fold or 10¹⁸-fold in the cell suspension compared to the concentration in the starting blood cell suspension.

In preferred embodiments where the analyte to be reduced is a small molecule, the methods of the invention are used to reduce the concentration of the analyte to a pharmacologically, immunologically or toxicologically inactive level. In preferred embodiments where the analyte to be reduced is an anti-pathogenic agent, e.g. an ethyleneimine oligomer, a phenothiazine derivative, a psoralen, an acridine derivatives, riboflavin it is preferred that methods of the invention are used to reduce the concentration of the anti-pathogenic agent to a level that is below the level that is liable to act upon the body.

In some preferred embodiments, the final reduced concentration of anti-pathogenic agent is less than about 1 g/ml, and preferably less than about 1 mg/ml. In additional preferred embodiments, the final concentration of the reduced anti-pathogenic agent is less than about 10⁻⁴ g/ml, less than about 10⁻⁵ g/ml, less than about 10⁻⁶ g/ml, less than about 10⁻⁷ g/ml, less than about 10⁻⁸ g/ml, less than about 10⁻⁹ g/ml, less than about 10⁻¹⁰ g/ml, less than about 10⁻¹¹ g/ml, or less than about 10⁻¹² g/ml.

In some embodiments, the washing procedure is automated. In some embodiments, washing is performed in a closed system to avoid introduction of environmental microorganisms.

In some embodiments the washing procedure follows a pretreatment of the blood cell suspension with a pathogen inactivation compound such as an ethyleneimine oligomer or derivative thereof.

In some embodiments, the blood cell suspension is run through a biocompatible filter prior to or following washing, preferable a leukoreducing filter.

In a preferred embodiment where the analyte to be reduced is a cell, e.g. a leukocyte, the blood cell suspension is treated with an ethyleneimine oligomer, e.g. dimer, trimer, or tetramer, or a derivative thereof, followed by the washing procedure of the invention. The cell analyte is reduced at least 100 fold, 10³-fold, 10⁴-fold, or 10⁵-fold in the cell suspension relative to the cell analyte concentration in the starting cell suspension. In more preferred embodiments, the cell analyte concentration is reduced at least 10⁶-fold, 10⁷-fold, 10⁸-fold, 10⁹-fold, 10¹⁰-fold or 10¹¹-fold in the cell suspension. Where the cell analyte to be reduced is a leukocyte, the above embodiment of the invention is based in part on the discovery that the combination of treating a red blood cell suspension with an ethyleneimine oligomer and washing according to the methods of the invention results in red blood cell suspension in which leukocytes have been substantially removed.

In another preferred embodiment where the analyte to be reduced is a leukocyte, a red blood cell suspension is, treated with an ethyleneimine oligomer, leukoreduced by filtration, and washed according to the procedure of the invention. The leukocytes are reduced at least 10³-fold, 10⁴-fold, or 10⁵-fold in the cell suspension by the above-described methods of the invention. In more preferred embodiments, the leukocyte concentration is reduced at least 10⁶-fold, 10⁷-fold, 10⁸-fold, 10⁹-fold, 10¹⁰-fold or 10¹¹-fold in the cell suspension.

In a preferred embodiment where the analyte to be reduced in a blood cell suspension is a prion protein, the amount of prion protein is reduced at least 10 fold, preferably 10²-fold relative to the amount of the prion protein in the starting blood cell suspension by the methods of the invention. Preferably, prion protein (PrP) is reduced at least 10³-fold, 10⁴-fold or 10⁵-fold relative to the amount of the prion protein in the starting blood cell suspension. More preferably, washing is sufficient to reduce the amount of the prion protein at least 10⁶-fold, 10⁷-fold or 10⁸-fold relative to the amount of the prion protein in the starting blood cell suspension. More preferably, the prion protein is reduced at least 10⁹-fold or 10¹⁰-fold relative to the amount of prion protein in the starting blood cell suspension.

In one embodiment of the invention, prion protein, is reduced in a blood cell suspension by the washing procedures of the invention. In another embodiment, prion protein is reduced in a blood cell suspension by the washing procedures of the invention wherein the wash solution comprises a lipophilic emulsion. In another embodiment of the invention, prion protein is reduced in a blood cell suspension by the washing procedures of the invention in combination with running the blood cell suspension through a blood compatible filter, preferably a leukoreducing filter. In another preferred embodiment, prion protein is reduced in a blood cell suspension by the wash procedures of the invention wherein the wash solution comprises a lipophilic emulsion and the blood cell suspension is run through a blood compatible filter.

In a preferred embodiment, the prion protein removed and/or reduced by the above methods of the invention is a pathogenic prion protein. While not wishing to be bound by theory, it is believed that blood and/or blood products may in some cases transmit prion pathogenic agents. In particularly preferred embodiments the prion protein removed and/or reduced by the above methods of the invention is an endogenous blood borne prion protein. In particularly preferred embodiments, the prion protein removed and/or reduced by the methods of the invention is a pathogenic blood-borne prion protein. In a particularly preferred embodiment, the pathogenic blood-borne prion protein is removed from a mammalian red blood suspension, particularly from mammalian (e.g. human) whole blood or red cell concentrate.

In a preferred embodiment, where the prion protein to be reduced and/or removed in a blood cell suspension comprises soluble prion protein, the soluble prion protein may be reduced by the washing procedures of the invention. In a preferred embodiment where the prion protein to be reduced and/or removed in a blood cell suspension comprises a membrane-associate prion protein, a lipophilic emulsion may be added to the washing buffer of the invention and/or the blood cell suspension may be run through a blood compatible filter. In preferred embodiments where the prion protein is reduced and/or removed from the blood cell suspension comprises an insoluble prion protein, a lipophilic emulsion may be added to the washing buffer of the invention and/or the blood cell suspension may be run through a blood compatible filter. In preferred embodiments where the prion protein to be reduced and/or removed from the blood cell suspension comprises multiple physical forms of prion protein a combination of washing, filtration and/or lipophilic emulsion can be used to achieve the above described log reductions.

In preferred embodiments, the blood cell suspension is assayed for the presence or absence of prion protein prior to and/or following washing procedures or wash/filter combinations of the invention. In particularly preferred embodiments, a red blood cell suspension is assayed for the presence or absence of pathogenic prion protein and/or aggregates following the washing or wash filter combinations of the invention. Detection of residual prion protein can follow an optional concentration step for concentrating prion protein, if any, remaining associated with the red blood cell composition following the wash procedures or wash/filter combinations of the invention.

In a particularly preferred embodiment, transmission or the risk of a prion mediated disease, particularly a transmissible spongiform encephalopathy, by a blood product is reduced. In another particularly preferred embodiment, the onset of a prion mediated disease, particularly a transmissible spongiform encephalopathy, is significantly delayed from the time of potential exposure via a blood product. In a preferred embodiment, reduction of the risk of transmission or delay in the onset of a prion mediated disease, particularly a transmissible spongiform encephalopathy, is provided by the following methods of the invention. Washing or washing and filtering a blood cell suspension according to the methods of the invention, thereby reducing the concentration of extracellular protein, preferably prion protein, particularly pathogenic prion protein. Transfusing the washed blood cell suspension to a recipient. In a particularly preferred embodiment, the recipient is a human recipient and the washed blood cell suspension is a human blood cell concentrate, such as a RBCC.

The method of the invention optionally comprises the step of detecting the reduction in concentration of extracellular protein. Detection in the reduction of extracellular protein may comprise detecting a reduction in the concentration of extracellular IgG, serum albumin, prion protein and/or pathogenic prion protein.

In preferred embodiments, the washed blood cell suspension transfused to a recipient comprises an extracellular protein concentration that correlates to that of a second washed blood cell unit where the second washed blood cell unit has been tested for infectious prion protein in a bioassay. In particularly preferred embodiments, the second washed blood cell unit results in a lower incidence of onset of a prion mediated disease, particularly a transmissible spongiform encephalopathy, in an animal bioassay compared to the incidence observed for an unwashed control. In another preferred embodiment the second washed blood cell unit results in a delayed onset of a prion mediated disease, particularly a transmissible spongiform encephalopathy in an animal bioassay compared to that observed in the bioassay for the unwashed control. In particularly preferred embodiments the washed blood cell suspension to be transfused comprises an extracellular IgG, serum albumin, prion protein and/or pathogenic prion protein concentration correlated to that of a blood cell unit that does not result in onset of a prion mediated disease, particularly a transmissible spongiform encephalopathy in a bioassay or results in delayed onset of a prion mediated disease, particularly a transmissible spongiform encephalopathy, in a bioassay.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based in part on the unexpected discovery that large volumes of red blood cells retain structural, metabolic and functional properties following extensive washing in saline solutions, particularly phosphate buffered saline solutions. The washed blood cells retain their properties following prolonged storage. The washed blood cells of the invention are suitable for transfusion.

The methods of the invention can be used to remove and/or reduce analytes generally from biological compositions. By “biological composition” is meant a composition containing cells or a composition containing one or more biological molecules, or a composition containing both cells and one or more biological molecules. Cell-containing compositions include, for example. blood, red blood cell concentrates, platelet concentrates, leukocyte concentrates, blood plasma, platelet-rich plasma, cord blood, semen, bone marrow, placental extracts, mammalian cell culture or culture medium, products of fermentation, and ascites fluid. Biological compositions may also be cell-free and contain at least one biological molecule.

By “biological molecule” is meant any class of organic molecule normally found in living organisms, including, for example, macromolecules such as nucleic acids, polypeptides, post-translationally modified proteins (e.g., glycoproteins), polysaccharides, and lipids. Biological molecule-containing biological compositions include, for example, serum, blood cell proteins, blood plasma concentrate, blood plasma protein fractions, purified or partially purified blood proteins or other components, a supernatant or precipitate from any fractionation of the plasma, purified or partially purified blood components (e.g., proteins or lipids), milk, urine, saliva, a cell lysate, cryoprecipitate, cryosupernatant, or portion or derivative thereof, and compositions containing products produced in cell culture by normal or transformed cells.

The biological composition can be from any desired animal. For example, the method can be used with cell suspensions containing mammalian blood cells (including human, non-human primate, canine, feline, equine, or rodent blood cells). Preferred mammalian blood cells are red blood cells or platelets.

A “blood product” as herein relates to a washed biological composition, which is therapeutically useful, e.g. for transfusion to a recipient.

A “prion mediated disease” as defined herein relates to a disease associated with the finding of abnormal prion protein and includes transmissible spongiform encephalopathy such as scrapie, bovine spongiform encephalopathy, Creutzfeldt-Jakob disease (CJD), and variant Creutzfeldt-Jakob disease.

In general, any wash solution that is osmotically compatible and non-toxic with the cell type being washed can be used. For blood cells, preferred wash solutions include saline (0.9% sodium chloride), phosphate-buffered saline (0.9% sodium chloride, 13 mM sodium phosphate or 0.9% sodium chloride, 30 mM sodium phosphate) and dextrose-saline (0.2% dextrose, 0.9% sodium chloride).

When red blood cells are washed according to the methods of the invention, washing is preferably performed so that the hematocrit of the washed red blood cells after completing the washing process is between 50 and 70%. Preferably, less than 20% red cells are lost as a result of the washing process

As discussed above, analytes can include small molecules. By “small molecule” is meant a molecule with a molecular weight of less than 1000 Daltons. Examples of the foregoing may include glycerol, DMSO, ethyleneimine oligomer, psoralens, phenothiazine-based agents, acridine-based agents, riboflavin or a drug which may include any drug which is recognized by American Association of Blood Banks or the FDA or the U.S. military as being a disqualification for donating.

As discussed above, analytes can also include a molecule larger than 1000 Daltons. For example, the analyte can be a macromolecule such as a nucleic acid or protein. Examples of protein analytes whose levels in blood cell suspension are reduced according to the methods of the inventions are prion proteins, particularly pathogenic prion protein. Other examples of analytes that are removed by the methods of the invention can include, cells, e.g. leukocytes, microbial pathogens (such as bacteria, fungal or protozoan organism), or infectious viral agents.

The term prion is a synonym for the infectious agent which causes transmissible spongiform encephalopathies—for example, human variant CJD, cattle BSE, and scrapie in sheep. The method of the invention can be used to remove and/or reduce the amount of any form of prion protein in biological compositions, particularly in blood cell suspensions. The term prion protein (PrP), as used herein includes the naturally-occurring, non-infectious forms (generically known as PrP^{C}), the pathogenic forms (generically known as PrP^{Sc}), β-folded forms, those PrPs produced in bacteria or eukaryotic cells by recombinant DNA techniques (generically known as recombinant PrP or recPrP) and re-folded in vitro into forms with predominantly ∝-helical (∝-recPrP) or β-(β-recPrP) secondary structure, or supra-molecular aggregates of one or a combination of these forms (aggregated PrP or PrP^{AG}). The term prion protein as used herein includes any physical form of PrP, e.g. PrP characterized by being soluble, insoluble, protease-sensitive, protease-insensitive, membrane-associated, not membrane-associated, aggregated or not aggregated. Pathogenic protein as used herein is used in the broad sense of an infectious protein and/or a simple product of disease. Pathogenic prion proteins of the invention, therefore, include any of the foregoing proteins that are infectious and/or are products of disease.

Prion protein, including pathogenic prion protein, further including blood borne pathogenic prion protein may be detected in a variety of ways including using one or more of the following methods: using antibodies, see, e.g. U.S. Pat. No. 5,846,533; the DELFIA® assay, see, Hemmila, Scand. J. Clin. Lab. Invest., 48:389-399, 1988, and MacGregor, et al., Vox Sang., 77:88-96, 1999; nucleic acid molecules, see, e.g. WO 97/15685; using an animal bioassay, see e.g. (Crozet, C., Flamant, F., Bencsik, A., Aubert, D., Samarut, J., and Baron, T. (2001). Efficient transmission of two different sheep scrapie isolates in transgenic mice expressing the ovine prp gene. J Virol 75, 5328-34; Manson, J. C., Barron, R., Jamieson, E., Baybutt, H., Tuzi, N., McConnell, I., Melon, D., Hope, J., and Bostock, C. (2000). A single amino acid alteration in murine PrP dramatically alters TSE incubation time. Arch Virol Suppl 95-102; Scott, M. R., Will, R., Ironside, J., Nguyen, H. O., Tremblay, P., DeArmond, S. J., and Prusiner, S. B. (1999). Compelling transgenetic evidence for transmission of bovine spongiform encephalopathy prions to humans. Proc Natl Acad Sci USA 96, 15137-42. Moore, R. C. and Melton, D. W. (1997). Transgenic analysis of prion diseases. Mol Hum Reprod 3, 529-44. Race, R. E., Priola, S. A., Bessen, R. A., Ernst, D., Dockter, J., Rall, G. F., Mucke, L., Chesebro, B., and Oldstone, M. B. (1995). Neuron-specific expression of a hamster prion protein minigene in transgenic mice induces susceptibility to hamster scrapie agent. Neuron 15, 1183-91; Telling, G. C., Scott, M., Hsiao, K. K., Foster, D., Yang, S. L., Torchia, M., Sidle, K. C., Collinge, J., DeArmond, S. J., and Prusiner, S. B. (1994). Transmission of Creutzfeldt-Jakob disease from humans to transgenic mice expressing chimeric human-mouse prion protein. Proc Natl Acad Sci USA 91, 9936-40.; 7. Westaway, D., Mirenda, C. A., Foster, D., Zebarjadian, Y., Scott, M., Torchia, M., Yang, S. L., Serban, H., DeArmond, S. J., Ebeling, C., et al (1991). Paradoxical shortening of scrapie incubation times by expression of prion protein transgenes derived from long incubation period mice. Neuron 7, 59-68; Prusiner, S. B., Scott, M., Foster, D., Pan, K. M., Groth, D., Mirenda, C., Torchia, M., Yang, S. L., Serban, D., Carlson, G. A., et al (1990). Transgenetic studies implicate interactions between homologous PrP isoforms in scrapie prion replication. Cell 63, 673-86.), using a cell based bioassay, see, e.g., Birkett, C. R., Hennion, R. M. Bembridge, D. A., Clarke, M. C., Chree, A., Bruce, M. E., and Bostock, C. J. (2001). Scrapie strains maintain biological phenotypes on propagation in a cell line in culture. EMBO J 20, 3351-8; Vilette. D., Andreoletti, O., Archer, F., Madelaine, M. F., Vilotte, J. L., Lehmann, S., and Laude, H. (2001). Ex vivo propagation of infectious sheep scrapie agent in heterologous epithelial cells expressing ovine prion protein. Proc Natl Acad Sci USA 98, 4055-9); and according to the examples described herein.

Onset of prion mediated disease, including transmissible spongiform encephalopathy, in animal bioassays may be detected by observing the animals for clinical signs of disease. For example, clinical signs of an ovine transmissible spongiform encephalopathy are variable but can include generalized neurological dysfunction, behavioral changes, nervousness, ataxia, puritis and poor conditioning. These signs can develop over a period of hours, days or weeks and experimental animals require regular attention on a day-to-day basis. Fallen stock should be regarded as potential victims of disease even if no previous clinical signs have been observed. Suspect cases can be confirmed by post-mortem examination of brain pathology for the pathogonomic triad of TSE lesions—vacuolation of the neuropil, hypertrophy and hyperplasia of glial cells and neuronal loss. In some cases, visible deposits of amyloid can be also seen under the fluorescent microscope. Immunohistochemical and Western blot screening of several discrete brain sections and sections of peripheral lymphoid tissues for the presence of abnormal prion protein are recommended to confirm TSE disease.

Without being bound by any particular theory, it is postulated that infectious particles in contaminated blood cell preparations can range in size from high order fibrillar aggregates to an abnormally folded monomeric protein. Accordingly, prions can be i) present in the surrounding fluid, ii) non-covalently attached to the erythrocyte surface by ionic or hydrophobic interactions, or iii) partially integrated into the RBCC membrane via its GPI membrane anchor. Therefore, it is postulated that prion reduction can be achieved according to the invention by exhaustive washing where continuous reduction of ambient PrP^{Sc} shifts association equilibria away from the RBCC surface.

One of the advantages of the invention is that the washed blood cells contain significantly lower levels of the analyte as compared to the corresponding unwashed cell suspension.

Another advantage of the invention is that it enables washing of large volumes of biological suspensions, such as blood cells. Thus, in some embodiments, the blood cells are provided in a suspension with a volume of at least 50 milliliters. In other embodiments, the suspension is provided in a volume of at least 100 mL, 125 mL, 250 mL, 400 mL, or even 1 L or more.

Blood cell suspensions used in the methods of the invention can include nucleated or a-nucleated cells. By an “a-nucleated cell” is meant a cell which, when mature, lacks a nucleus. Examples of a-nucleated cells are platelets and red blood cells. Because blood transfusions typically involve transfer of a-nucleated cells, it is can be desirable to separate these cells from other blood components, such as white blood cells (e.g., lymphocytes, neutrophils, and monocytes) and biological molecules (e.g., albumin, immunoglobulins, clotting factors and complement). For example, prior to transfusion, whole blood may be separated into the red blood cell portion (containing a small portion of white blood cells) and plasma (which also contains a small percentage of the white blood cells). Standard methods, such as a Ficoll or Percoll gradient can be used to accomplish the separation different components of whole blood based on differences in their density. Numerous systems for accomplishing the separation of a-nucleated cells are commercially available and include the MCS®⁺ Apheresis system from Haemonetics Corp. (Braintree, Mass.). The above described blood cell suspensions may be washed according the methods of the invention.

In a preferred embodiment, analytes are removed from and/or reduced in a biological composition using centrifugation. For example, the method can include centrifuging the blood cells to form a packed cell fraction and a supernatant that includes the extracellular fluid. The supernatant is then removed from the packed cell fraction. Washing solution is then added to the packed cell fraction and the packed cell fraction is resuspended in the washing solution to form a resuspended cell suspension. The resuspended cells can be recentrifuged and resuspended. In some embodiments, the cells are centrifuged and resuspended 2, 3, 4, or 5 or more times as described herein. The present invention is not limited to a particular number of washes rather the number of centrifugation and resuspension steps performed will depend on the extracellular fluid fold reduction or extracellular fluid/analyte fold reduction desired and the ratio of wash solution to extracellular fluid. Centrifugation systems which may be used with the invention and materials, including disposable sets for use with the centrifugation systems are commercially available, and may include Haemonetics V215 Centrifuge (Braintree, Mass.), CS-30000 and Amicus from Baxter (Deerfield, Ill.), Spectra from Gambro (Arvada, Colo.), Cobe 2991 cell processor from Gambro (Arvada, Colo.).

The washing methods of the present invention, may occur at a temperature between 1° C. and 40° C., preferably, between 20-30° C., or more preferably at room temperature. Centrifugation speed may be 5,000 to 11,000 rpm, preferably 6,000 to 10,000 rpm.

The washing steps can be either manual washings performed under sterile conditions, or automated washings performed under sterile conditions. For example, the RBCs may be in a sterile container, such as a plastic bag. The bag is then attached to a machine that can, under sterile conditions, pump the cells out of the bag, optionally rinse the bag with wash solution, dilute the RBCs with sterile wash solution, gently mix the solution for a desired time at a desired temperature, collect the red blood cells by centrifugation, discard the used wash solution (e.g., saline, dextrose-saline, phosphate buffered saline). A new wash solution is then added and the wash and centrifugation steps are repeated for a desired number of times. After the final round of washing, the cells can be resuspended in storage solution and returned to the original container. The cells can then be used immediately, stored, or frozen as desired.

Where the cells are to be stored up to seven days, the storage solution may be for example, dextrose-saline, saline, or phosphate-buffered saline. Where the cells are to be stored more than seven days at about 4° C., the storage solution is a nutritive storage solution comprising glucose and phosphate, such as NUTRICEL® [AS-3] from Pall Corporation; AS-1 from Baxter (Deerfield), AS-5 from Terumo, CPDA-1, CPD, CP2D from Pall Corporation. Where the cells are to be stored frozen, the storage solution comprises glycerol or DMSO.

Where the washing method of the invention is automated the blood cell suspension may be pumped to the centrifuge in suitable tubing. The pump rate and tubing size is selected so as to minimize cell damage and total washing time while maximizing pump efficiency. The pump rate is preferably not, however, dynamically adjusted to avoid the risk of osmolarity shock. Accordingly, the pump rate of the invention may comprise between 50 and 200 mL/min. Disposable blood tubing sets are manufactured typically from medical grade polyvinyl chloride and may be purchased from multiple commercial source, e.g. Pall Corp. East Hills, N.Y., Baxter International, Deerfield, Ill., Haemonetics, Braintree, Mass. and Cobe, Arvada, Colo.

The blood cell suspension can be run through a blood compatible filter, preferably a leukoreducing filter. Commercial blood leukocyte reduction filters for red blood cells and whole blood which are capable of reducing the level of white blood cells by >99.9% (>3 log reduction) are available from the following companies Pall Corporation, East Hills, N.Y.; Hemasure, Marlborough, Mass.; and Baxter Healthcare, Deerfield, Ill.

A lipophilic emulsion can be added to the wash solution of invention, particularly where the analyte to be removed and/or reduced in blood cell suspension is a prion protein or a lipid enveloped virus. Such a lipophilic emulsion can be composed of the same composition as those used for intravenous nutritional purposes (reviewed in Advances in intravenous lipid emulsions. Carpentier Y A, Dupont I E, Deloyers World J Surg 2000 December;24(12):1493-7) which are normally formulated at a 10% to 20% composition of long chain triglycerides (LCT) emulsion. Medium chain triglycerides in a stable emulsion may also be suitable for enhancing washout of lipid soluble products from blood. The lipid emulsion facilitates the removal of lipophilic soluble agents from the blood by the lipid emulsion acting as a non-hemolytic solvent. The preferred concentration of lipid emulsion in the wash solution to facilitate removal of noxious lipophilic substances from the blood, ranges from 0.1 to 20%.

Washing is preferably performed in a closed system, e.g., a functionally closed system. Washing in a closed system allows for storage of cells for prolonged periods (e.g. more than 1, 7, 14, 21, 28, 35, 40, 42, or even 50 days) after washing without risk of having introduced environmental contaminants, such as microbial contaminants.

Washing may be completed in 30 minutes to 5 hours and may require more than 4 liters of wash solution. In preferred embodiments, washing is completed in less than 4 hours and use less than 10 liters of wash solution. In particularly preferred embodiments washing is completed in 30 to 60 minutes and uses more than three but no more than six liters of wash solution. In particularly preferred embodiments, 140 to 260 mL, preferable 200 to 220 mL of red blood cells having a hematocrit of 40-98% are washed in five to five and a half liters of wash solution for 170 to 195 minutes.

In one embodiment, whole blood is diluted with nonbuffered sterile saline (i.e., 0.9% NaCl), and the cells are concentrated by centrifugation to isolate the RBC component. The RBC component is then resuspended in sterile saline and allowed to mix (under gentle mechanical agitation) for 10 minutes at 22° C. The washing and resuspending procedure is repeated until a desired log removal of an analyte has been achieved. A similar procedure is used for washing isolated platelets.

An additional method for cell washing includes a hollow fiber dialysis, where separation of soluble materials from red cells is achieved by recirculating red cells through a hollow fiber with pores sufficiently small to retain red cells but large enough to allow macromolecules to pass (0.2-1 micron pore). The extra-capillary chamber is continuously flushed with wash fluid, which serves to replace and remove the extra-cellular soluble materials diffusing across the hollow fiber walls. The equipment and materials can be obtained from, for example, Mission Medical (San Francisco), Baxter (Deerfield), Gambro (Lund, Sweden) and Asahi (Tokyo, Japan).

Alternatively, a spinning membrane is used. Separation of extra-cellular soluble materials from red cell concentrates is achieved using a porous membrane with pores sufficiently small to retain red cells but large enough to allow soluble molecules to exit through (0.2-1 microns). The membrane is housed as a hollow cylinder into which red cells are introduced while the cylinder is rotating. The rotating membrane allows red cell extracellular fluid to be removed by passing through the membrane. Commercial companies include Nexell (Santa Ana, Calif.), and the Fenwal Division of Baxter Healthcare provides a plasmapheresis device (Auto C System), which is based on the spinning membrane approach.

If desired, agents that enhance the stability of blood cells or themselves inactivate or remove infectious agents can be used in the washing procedures. Examples of such agents are antimicrobial agents and antiviral agents. Examples include ethyleneimine oligomers, such as dimers, trimers and tetramers and derivatives thereof. Ethyleneimine oligomer inactivating agents are preferably used prior to washing a-nucleated blood cells such as red blood cells or platelets. Where ethyleneimine oligomers are used with, prior to or during the wash procedures of the invention, it is preferred that the wash solution not comprise a quenching agent. Ethyleneimine oligomer and methods of using them in biological compositions are described in WO00/18969 and WO98/51660.

It has been unexpectedly found that red blood cells washed according to the methods of the invention show prolonged in vitro or in vivo survival, or both. For example, in some embodiments, the red cells maintain their in vivo viability in the body following washing and, optionally prolonged storage, such that >75% of the washed cells remain in the circulation 24 hours after their transfusion. Viability can be measured using methods known in the art (for example, using ⁵¹Cr radiolabelling method), and as described below in the Examples. Red cells washed according to the methods of the invention show prolonged in vitro stability. For example, in some embodiments, cells in blood cell preparation washed according to the invention achieve a shelf life of at least 7 days. In some embodiments, washed blood cells have shelf lives of 14, 21, 28, 40, or 42 days or more. Preferably, the mean hemolysis level of blood cells washed according to the invention and stored for prolonged periods is less than, e.g., 5%, 2.5%, 1%, or even 0.5%. Preferably ATP levels are maintained above 1.5 μg/mol.

The invention will be further illustrated in the following non-limiting examples.

### EXAMPLE 1

### Removal of Analytes from a Red Blood Cell Suspension

1A. 5000 mL Red Blood Cell Wash Procedure

The red blood cell suspension, which may be leukoreduced, is washed by an automated system under sterile conditions in a closed system. The wash procedure is carried out at room temperature. The procedure is performed in the Haemonetics V215 (Haemonetics, Braintree, Mass.). The centrifuge bowl size is 275 mL or 325 mL. The RBCC wash cycle uses approximately 5000 mL of saline and takes about 190 minutes to complete.

Anti coagulated red blood cell concentrate comprising a volume of 280-400 mL at a hematocrit of 50-70 is placed in an incubation bag and connected to the V215 aseptically. First, 300 mL of wash solution (e.g., 0.2% dextrose, and 0.9% saline) is added to the incubation bag, and allowed to equilibrate for 45 seconds. The contents of the incubation bag are then pumped into the centrifuge bowl, which is spinning at a speed of 8000 rpm. The pump rate is between 50 to 200 mL/min. The incubation bag is rinsed with 80 mL of wash solution, and the rinse is transferred to the bowl. The bowl is next rinsed with 50 mL of wash solution. The bowl contents are returned to the incubation bag, diluted with 300 mL of wash solution and allowed to equilibrate for 45 seconds. The contents of the incubation bag are transferred to the bowl. A second incubation bag flush with 80 mL of wash solution is next performed. The second flush is added to the bowl. The bowl is rinsed with a second bowl rinse of 50 mL of wash solution. The bowl contents are returned to the incubation bag and a third dilution of 300 mL of wash solution is added and equilibrated for 45 seconds. The bag and bowl are rinsed as above. The above-described procedure (dilute, flush, rinse, return) is repeated ten more times. During the 12^{th} round, the dilution step is as described above but after transfer to the bowl the bowl is stopped and the 95 mL wash cycles begin.

Thirty mL of the bowl contents is transferred to the incubation bag, and after a 10 second delay, the centrifuge is restarted, and the 30 mL of suspension from the incubation bag is returned to the bowl. The bowl is spun for 30 seconds at 8000 rpm, and 95 mL of wash solution is transferred to the bowl. The centrifuge bowl again stops and again 30 mL of the bowl contents is transferred to the incubation bag, and after a 10 second delay, the centrifuge is restarted and the 30 mL of suspension from the incubation bag is returned to the bowl. The bowl is spun for 30 seconds at 8000 rpm. This procedure is repeated three to five additional times. After the seventh wash, 240 mL of storage solution is added to the centrifuge bowl with the red blood cells and the contents of the bowl are transferred to a final product bag. The final product bag is removed and sealed.

Thus, the washing procedure consists of twelve 300 mL dilutions and 7 washes, requiring a total of about 5 L of wash solution and 190 minutes of washing.

1B. 5500 ml Red Blood Cell Wash Procedure

A red blood cell suspension is washed by an automated system under sterile conditions in a closed system. The wash procedure is carried out at room temperature. The procedure is performed in the Haemonetics V215 (Haemonetics, Braintree, Mass.). The centrifuge bowl size is 275 mL or 325 mL. The RBCC wash cycle uses approximately 5500 mL of saline and takes about 190 minutes to complete.

Anti coagulated red blood cell concentrate typically comprising a volume of 250-450 mL at a hematocrit of 50-70 is placed in an incubation bag and connected to the V215 aseptically. To begin the procedure the line to the final product bag is primed with 100 mL of wash solution. This wash solution is used during the process to periodically flush with 5 mL of wash solution the tubing T-junction shared by the inlet line, line to final product bag, and line to blood pump. This T-junction flush serves to prevent analyte from contaminating the line to the final product bag. To initiate the pre-dilution sequence, the contents of the incubation bag are pumped into the centrifuge bowl, which is spinning at a speed of 8000 rpm. The pump rate is between 50 to 200 mL/min. Once the incubation bag is empty, the pump reverses and delivers 150 mL of wash solution into the incubation bag as a flush volume. During the delivery of the flush volume the incubation bag is agitated (180 hz, 1.5 inch peak-to-peak amplitude) by a shaker table tilted at 5.5 degrees from true horizontal. Once the 150 mL is delivered to the incubation bag, the shaker remains on for about 45 seconds and then stops. The flush volume is then emptied from the incubation bag and pumped into the centrifuge bowl. The T-junction is then flushed (T-flush) with 5 mL of wash solution, pumping out of the final product bag line and into the centrifuge bowl. Following the T-flush, the line to the donor pressure monitor (DPM) is purged of fluid that migrated into this line during the previous steps. This is accomplished by opening a purge valve internal to the DPM and drawing (pumping) approximately 8 mL of air through the DPM line's antibacterial filter to draw the fluid residing in the DPM line into the bowl. This DPM line purge occurs periodically throughout the process to prevent trapping of analyte in the line to the DPM where it can contaminate the process fluids at later stages of processing. After the DPM line purge, the centrifuge is braked to a stop. Once the centrifuge bowl is no longer spinning, approximately 30 mL of the bowl contents are pumped back into the incubation bag. The contents of the bowl are then allowed to equilibrate for 30 seconds before the centrifuge restarts and accelerates to 8000 rpm. The 30 mL in the incubation bag is returned to the centrifuge bowl at 50 to 100 mL/min. The pump then stops while the centrifuge remains spinning. After 15 seconds, the incubation bag shaker starts and the pumps deliver a flush volume of 150 mL of wash solution to the incubation bag. The shaker remains on for approximately 45 seconds and then stops. The flush volume is then pumped out of the incubation bag and into the spinning centrifuge bowl. The T-flush and the DPM line purge (as described above) are repeated. The contents of the bowl are then rinsed with approximately 130 mL of wash solution by pumping wash solution into the bowl at a rate of approximately 50 mL/min. The centrifuge is then stopped and the contents of the bowl are returned to the incubation bag. This completes the pre-dilution sequence of the process. The dilution sequence follows.

To begin the dilution sequence, the shaker starts and 300 mL of wash solution (e.g., 0.2% dextrose, and 0.9% saline) is added to the incubation bag diluting the incubation bag contents. The shaker stops 10 seconds later, and the incubation bag contents are allowed to equilibrate for 45 seconds. The contents of the incubation bag are then pumped into the centrifuge bowl, which is spinning at a speed of 8000 rpm. The pump rate is between 50 to 200 mL/min. The T-flush and DPM line purge are repeated. The incubation bag is flushed with 80 mL of wash solution, agitated on the shaker table for 30 to 45 seconds, and then the flush volume is transferred to the bowl. The bowl is next rinsed with 50 mL of wash solution at a pump rate of 50 to 100 mL/min. The bowl contents are returned to the incubation bag. Once the bowl is emptied, the contents of the line to the system pressure monitor (located off the effluent line from the bowl) are purged into the bowl. This occurs to purge the system pressure monitor (SPM) line of the fluid that migrated into this line during the previous steps. This is accomplished by opening a purge valve internal to the SPM and drawing (pumping) approximately 8 mL of air through the SPM line's antibacterial filter allowing the fluid residing in the SPM line to be drawn into the bowl. This SPM line purge occurs periodically throughout the process to prevent trapping of analyte in the line to the SPM where it can contaminate the product at later stages of processing. The SPM purge volume is emptied from the bowl into the incubation bag.

The incubation bag contents are then agitated by the shaker, diluted with a second dilution volume of 300 mL of wash solution, and allowed to equilibrate at rest for 45 seconds. The contents of the incubation bag are then transferred to the bowl. The T-flush and DPM line purge are repeated. A second incubation bag flush with 80 mL of wash solution is next performed. The second flush is added to the bowl. The bowl is rinsed with a second bowl rinse of 50 mL of wash solution. The bowl contents are returned to the incubation bag. The SPM line purge is repeated. A third dilution of 300 mL of wash solution is added and equilibrated for 45 seconds. The contents of the incubation bag are returned to the bowl. The T-flush and DPM line purge are repeated. The bag and bowl are rinsed as above. The SPM line is purged. The above-described procedure (dilute. T-flush, DPM line purge, bag flush, bowl rinse, return, SPM line purge) is repeated ten more times. The DPM and SPM line purges occur during only the first 10 dilutions. The last T-flush in the 12^{th} dilution will completely empty the product bag line of the wash solution with which it was primed. During the 12^{th} round, the dilution step is as described above but after transfer to the bowl and the T-flush, the 95 mL wash cycle sequence begins.

First, 95 ml of wash solution is pumped into the bowl at a rate of 75 ml/min. The centrifuge will then stop and thirty mL of the bowl contents is transferred to the incubation bag, and after a 45 second delay, the centrifuge is restarted. The bowl is spun for 30 seconds at 8000 rpm and then the 30 mL of suspension from the incubation bag is returned to the bowl. A second 95 mL of wash solution is then transferred to the bowl. The centrifuge bowl again stops and again 30 mL of the bowl contents is transferred to the incubation bag, and after a 45 second delay, the centrifuge is restarted. The bowl is spun for 30 seconds at 8000 rpm and then 30 mL of suspension from the incubation bag is returned to the bowl. This procedure is repeated five additional times. After the seventh wash, the centrifuge is stopped and 30 mL of the bowl contents is emptied to the product bag. The centrifuge restarts at 8000 rpm and spins for 45 seconds. Then 250 mL of storage solution is added to the centrifuge bowl with the red blood cells at a rate of 75 ml/min. The centrifuge is stopped and the contents of the bowl are transferred to the final product bag. The final product bag is removed and sealed.

Thus, the washing procedure consists of a predilution sequence, twelve 300 mL dilutions and seven 95 mL washes, requiring a total of about 5.5 L of wash solution and a time period of 190 minutes.

### EXAMPLE 2

### In Vitro Biochemical Characterization and In Vivo Viability of Washed Red Blood Cells

Control blood units are collected from healthy human subjects in CP2D and leukoreduced via a Pall WBF2 leukoreduction filter at room temperature. Four hours after collection, the cells are converted to AS-3 Red Blood Cells (Medsep, Covina, Calif.) through a hard spin (5,000 g×5 min at 20° C.). Experimental units are collected into CPD and leukoreduced via a Sepacell RS2000 leukoreduction filter (Baxter, Round Lake, Ill.). After a 4 h room temperature hold, they are converted to packed cells by centrifugation at 1.615 g for 4 min at 20° C. to achieve a hematocrit of 75-80%. Ethyleneimine oligomer is then added aseptically to the unit at 0.1% v/v to achieve a concentration of approximately 920 μg/mL. Units are then washed with 6 L 5% dextrose/0.9% NaCl in a closed system device (Haemonetics 215, Braintree, Mass.) that has a bowl capacity of circa. 270 mL according to the procedure described in Example 1A. A saline solution of sodium thiosulfate is added to obtain a final concentration of 0.2 mM, and 100 mL of AS-3 are added to the unit.

Units are held in a monitored refrigerator at 1-6° C. for 42 days. Aliquots are taken for sampling via sterile connection device (SCD312, Terumo, Elkton, Md.) before and after processing and after 21, 28, 35 and 42 days of storage. Unit masses are converted to volumes using a calculated specific gravity: (1412*mass)/(spun hematocrit+1436). See, e.g., Halling et al, Transfusion 31:21S, 1991.

After 28 days of storage, an aliquot is taken for radiolabeling with ⁵¹Cr using standard techniques. See, e.g. The International Committee for Standardization in Hematology: Recommended methods for radioisotope red cell survival studies. Blood; 38:378-86, 1971; and Moroff et al., Transfusion, 24:109-114, 1984.

On the morning of the same day, a fresh sample is collected from the human subject into heparin for the determination of red cell volume simultaneously using ^{99m}Tc radiolabeling. See, Bandy et al., J. Nucl. Med.,16:435-437, 1975. Simultaneous injection of the two radiolabels is followed with multiple venous samplings to 30 min (to determine red cell volume via ^{99m}Tc), and at 24 h (to determine recovery via ⁵¹Cr). Concentrations of the ethyleneimine oligomer are determined on samples of lysed cells plus supernatant taken after addition of the chemical, at the end of the incubation period, after the washing step, and after 21 and 28 days of storage using HPLC having a sensitivity of 0.03 μg/mL.

Cell counts are performed by automated counters (Advia 120, Bayer, Norwood, Mass.) except for leukocyte enumeration after leukoreduction, which is performed via a Nageotte chamber. See, Dzik et al., Transfusion 33:272-273, 1993.

Supernatants from the units are spun twice at 3600 rpm (MP4R, International Equipment Company, Needham Heights, Mass.) for 10 min and then analyzed for hemoglobin using a Drabkin's reagent method (Sigma, St. Louis, Mo.) automated on the COBAS FARA (Roche, Nutley, N.J.) with a turbidity correction. Supernatant electrolyte concentrations are determined by ion-specific electrode (Hitachi 917, Boehringer Mannheim Corporation, Indianapolis, Ind.). Glucose is determined by glucose oxidase (Hitachi 917). Lactate is determined by lactate oxidase/peroxidase end point reaction (Hitachi 917). The pH is determined on a blood gas analyzer (Model 855, Bayer) and read at 37° C. A red cell perchloric acid extract is neutralized with 3M K₂CO₃ and analyzed for ATP (by measurement of NADH oxidation by glyceraldehyde phosphate dehydrogenase following use of ATP by phosphoglycerate phosphokinase) and DPG (by measurement of NADH oxidation) on the Cobas-FARA using adapted reagent kits (Sigma). Assays on supernatants are conducted in batches after storage of processed specimens for up to 4 months at −70-80° C. Biochemical assays are performed in duplicate with an averaging of results and repetition of duplicates with discrepant values.

Units are typed for ABO and Rh at the end of the storage period. At that time, they are also crossmatched against the subject's plasma at the antiglobulin phase using standard techniques. See, e.g. Technical manual, 13^{th} ed. Bethesda: American Association of Blood Banks, 1999.

Subjects undergo a battery of tests at entry into the study and before and after each reinfusion using standard methods of the medical center's clinical laboratory. These analyses include: complete blood count, urinalysis, serum electrolytes, phosphate, uric acid, BUN, creatinine, aspartate and alanine aminotransferases, lactate dehydrogenase, total bilirubin, glucose, alkaline phosphatase, total protein, albumin, triglycerides, and cholesterol.

Statistical analysis is conducted by paired t-test with a two-tailed probability of 0.05 selected as the criterion to reject the null hypothesis. All data are expressed as mean±1 standard deviation.

Ethyleneimine oligomer addition achieves the expected concentration: 920 μg/mL immediately after addition. Samples taken immediately after the washing step and at 21 and 28 days of storage are below the limit of detectability of the analytic system indicating that greater than a 4 log₁₀ reduction in concentration has occurred.

The difference in the handling of control and experimental units occurs because of the definition of the control unit that is assumed to provide assurance of the lack of a typical results on storage of red cells in these subjects rather than as a means to identify the effect of a particular feature of the experimental system. As a consequence, the spun hematocrit of the units is different (p<0.05) between control and experimental units (64.9±1.3 vs. 50.8±3.4%) at the start of the storage period the pH is slightly higher in the control group (6.76±0.02 vs. 6.52±0.05) on Day 0, and the total time to storage is shorter in the control group (5-6 h) as opposed to the experimental group (15-16 h). In addition, the control units have about 10-15% of the plasma remaining while almost no plasma remains in the experimental units after washing. All units are stored in polyvinyl chloride bags, but the control units are in bags provided by Medsep while the washed experimental units are in bags obtained through Haemonetics. All units have fewer than 1×10⁶ leukocytes.

For the treated, washed cells, the hematocrit falls from a level similar to that created through hard spin production of a “packed red cell unit” to that delivered from the Haemonetics 215. Recovery of red cells through the process, approximately 80%, is limited by the capacity of the instrument's bowl (275 mL total capacity). No hemolysis is noted visually. Changes in electrolytes, pH, and glucose parallel the content of the wash solution. ATP is maintained. DPG falls to approximately half of its initial concentration.

Glucose concentrations fall and lactate concentrations rise during storage. The rate of glucose consumption (control: 0.37±0.09 vs. 0.26±0.09 mmole/10⁶ red cells) approaches, but does not reach statistical significance, whereas lactate production (control: 0.91±0.12 vs. 0.42±0.09 mmole/10⁶ red cells) is higher in the control units. Supernatant potassium levels are lower in the experimental units. The difference in pH noted on Day 0 after washing continues throughout the storage period and is significantly different at Day 42.

Hemolysis remains below 1% in all units throughout the storage period. There is a trend toward increased hemolysis in the experimental units that is more evident with longer storage (at Day 42, control: 0.23±0.11 vs. experimental: 0.70±0.24%; p>0.05). ATP levels are not significantly different between groups throughout the storage period (at Day 42, control: 2.89±0.65 vs. experimental: 1.79±0.50 μmole/g Hb; p>0.05).

Red cells are reinfused into the subjects after 28 days of storage. The 24 h recoveries using the double-label technique are not different between control and experimental groups, as shown in Table 1.

TABLE 1
Recovery and Survival of Red Cells
24 h ⁵¹Cr Recovery
(Double label method)

| | |
|---|---|
| Experimental Units | 85.0 ± 5.0% |
| Control Units | 85.9 ± 2.7% |

### EXAMPLE 3

### Leukocyte Reduction

Ten units of anticoagulated whole blood units are collected and leukoreduced via a Pall BPF4B leukoreduction filter at 4° C. Nine units are washed according the procedure of Example 1A. Eleven units of the blood are treated with 0.1% v/v ethyleneimine oligomer, to achieve a concentration of approximately 920 μg/mL for 24 hours at 23° C. and washed according to the procedure of Example 1A.

Prior to leukofiltration, washing or ethyleneimine oligomer treatment followed by washing, the units of blood have between 2.4 to 4.7×10⁹ leukocytes per unit of blood. Leukofiltration alone reduces leukocyte content to between 0.4 to 56×10⁶ leukocytes per unit of blood. Washing alone reduces the leukocyte content to between 11 to 1100×10⁶ leukocytes per unit of blood. Ethyleneimine oligomer treatment and washing but without leukofiltration, reduces the leukocyte content to between 1.3 to 4.1×10⁶ leukocytes per unit of blood.

### EXAMPLE 4

### Comparison of Phosphate Buffered Wash Solution with Unbuffered Wash Solution

### 4A. Biochemical Parameters After Wash and a 42 Day Storage Period

Twelve identical pairs of standard anticoagulated, leukofiltered human RBCC units are treated for 24 hours at 23° C. with 0.1% (v/v) [920 μg/mL] ethyleneimine oligomer. The ethyleneimine oligomer is added to the RBCC units as a 2% v/v stock solution in 0.25 M filter-sterilized NaH₂PO₄. One treated unit from each pair is washed according to the procedure of Example 1A, with a phosphate buffered saline (PBS) wash buffer (0.9% NaCl, 12.5 mM Na-phosphate pH 7.7) and another with standard unbuffered saline (0.9% NaCl, 0.2% Dextrose, Baxter) All units are stored in AS-3 solution for 42 days at 1-6° C. After 42 days biochemical parameters are determined. The average values for units washed with PBS and Saline after 42 days of storage are: hemolysis 0.55±0.21% and 0.74±0.34%; ATP levels 3.28±0.61 μmole/gHgb and 2.33±0.44 μmole/g Hgb; extracellular K⁺ at 43±7.4 me/L and 40±2.8 respectively.

4B. Analyte Reduction

Seven sets of identical standard, anticoagulated, leukofiltered human RBCC units are treated for 24 hours at 23° C. with 1% (v/v), 920 μg/mL of ethyleneimine oligomer. The ethyleneimine oligomer is added to the RBCC units as a 2% v/v stock solution in 0.25 M filter-sterilized NaH₂PO₄. The treated units are washed according to the procedure of Example 1A or 1B with a saline wash solution (0.9% sodium chloride, 0.2% Dextrose), or a phosphate-buffered saline (PBS) having one of the following three compositions, 0.9% sodium chloride, 12.5 mM sodium phosphate (PBS), 0.9% sodium chloride, 50 mM sodium phosphate (PBS-50) or 0.9% sodium chloride, 75 mM (PBS-75) sodium phosphate. Following washing, the ethyleneimine oligomer concentration is determined by HPLC having a sensitivity of ≧30 ng/mL. Typically under experimental conditions used in saline washed RBCC 72 ng/mL residual ethyleneimine oligomer is detected, while 54 ng/mL is detected in RBCC washed with the 12.5 mM phosphate buffered solution, 36 ng/mL is detected in the RBCC washed with 50 mM phosphate buffered solution and 15 ng/mL is detected in the RBCC washed with the 75 mM phosphate buffered solution. All treated and washed units are stored at 1-6C for 42 days. The average values for units washed with Saline, PBS, PBS-50 and PBS-75 after 42 days of storage are: hemolysis 0.8±0.28%, 0.42±0.06%, 0.43±0.13% and 0.68±0.0.17%; ATP levels 2.05±0.06 μmole/gHgb, 3.42±0.18 μmole/gHgb, 3.67±0.33 μmole/g Hgb and 4.25±0.41 μmole/g Hgb; extracellular K⁺ at 38.5±5.1 mEq/L, 36.6±4.3 mEq/L, 35.7±3.1 mEq/L and 36.3±3.2 mEq/L respectively.

### EXAMPLE 5

### Assay for Removal of Protein Analyte from Washed Red Blood Cells

5A. Assay for Removal of Human Serum Albumin

A Western blot chemiluminescence assay is used to determine the level of protein removal by continuous and repetitive washing of red blood concentrates according to the method of Example 1A.

Each sample to be tested for the levels of human serum albumin (HSA) present is divided into separate aliquots. One aliquot (1-mL) of each sample is centrifuged at 2500 for 10 min and the supernatant is removed. Three samples labeled A, B and C from each set are analyzed for the presence and/or absence of albumin. Sample A refers to blood before treatment. Sample B is control blood-washed, and Sample C is blood treated with 0.1% (v/v), 920 μg/mL ethyleneimine oligomer for 24 hours at room temperature and then washed. The pre-treatment samples (Sample A) are diluted 1:10,000 prior to loading on an SDS gel. Samples B and C are not diluted. All samples are further diluted with 2×SDS gel reduced-sample buffer and boiled for 3 min.

Ten μl of each of the samples are separated by polyacrylamide gel electrophoresis and electrophoretically transferred to nitrocellulose membrane using the Bio-Rad semi-dry system. Nonspecific binding sites are blocked by rocking the membrane in blocking (3% Dry-Powder mild, made in 1×PBS) solution for 1 hour at room temperature (alternatively overnight at 4 degrees). The blot is incubated with human serum albumin-specific human monoclonal antibody (clone # HAS-11, Sigma, lot # 129H4847). Antibody is diluted in 1×PBS solution at 1:2000 dilution and placed in contact with the membrane. Following binding of the primary antibody, the membrane is washed with 1×PBS/Tween, twice for 5 minutes, followed by copious amounts of DD-water. The membrane is then incubated with a 1:30,000 dilution of Protein A-HRP conjugate and incubated for 45 min. The blot is rinsed one time for 5 minutes with 1×PBS and then with water. Visualization of the enzyme-labeled secondary antibody is accomplished with chemiluminescent detection method (ECL), using the Amersham Pharmacia solutions kit.

For preparation of standards, pure human serum albumin from Alpha Biotech (5%) is buffer exchanged into 5 mM Sodium Phosphate (pH 7.4). The protein concentration is 31.1 mg/mL.

The results indicate that using this method of detection, it is possible to detect as low as 10 ng of HSA in a sample. Each of seven samples tested, four show very similar albumin removal levels for both the control and treated samples. The levels of protein remaining are much lower than the lowest standard used, 10 ng.

The albumin concentration in normal human plasma is between 30-50 mg/mL, therefore the removal level of albumin according to the method described in Example 1A should be at least 6 logs.

5B. Assay for Removal of Human Serum Albumin and IgG

A Western blot chemiluminescence assay is used to determine the level of protein removal by continuous and repetitive washing of leukoreduced red blood concentrates according to the method of Example 1A.

Each sample is divided into separate aliquots. One aliquot (1-mL) of each sample is centrifuged at 5000×g for 5 minutes, the supernatants removed and recentrifuged at 16,000×g for 10 min and the supernatant is transferred away from the pellet. The samples are quantitatively analyzed for the presence of HSA and IgG. Sample A refers to blood before treatment. The pre-treatment samples (Sample A) are diluted 1:2000 or 1:10,000 prior to loading on an SDS gel for HSA and IgG analysis respectively. Post wash samples are not diluted. All samples are further diluted with 2×SDS gel non-reduced-sample buffer and boiled for 3 min.

Ten μl of each of the samples are separated by polyacrylamide gel electrophoresis and electrophoretically transferred to nitrocellulose membrane. Nonspecific binding sites are blocked by rocking the membrane in blocking (5% Dry-Powder mild, made in 1×TBST) solution for 1 hour at room temperature (alternatively overnight at 4 degrees). For analysis of HSA, the blot is incubated with human serum albumin (HSA)-specific human monoclonal antibody (clone # HSA-11, Sigma, A8763). Antibody is diluted in 1× blocking solution at 1:2000 dilution and placed in contact with the membrane. Following binding of the primary antibody, the membrane is washed with 1×PBS/Tween, 4 times for 5 minutes. The membrane is then incubated with a 1:10,000 dilution of sheep anti-mouse IGG HRP conjugate and incubated for 60 min. The blot is rinsed 4 times for 5 minutes with 1×TBST. For detection of IgG, the procedure is identical, except that only a 1:30,000 dilution of Protein A HRP conjugate (Pierce#32400) is used for detection. Visualization of the enzyme-labeled secondary antibody is accomplished with chemiluminescent detection method (ECL+, Pharmacia Amersham) Quantitation is made by capturing the image with a Flour S Chemiluminescent Imager (BioRad) and is analyzed using Quantity One software (BioRad).

For preparation of standards, pure human serum albumin, essentially immunoglobulin free from Sigma (A8763) is resuspended and diluted in PBS buffer to the desired concentrations. Pure human IgG (Alpha Biotech) was used as the immunoglobulin standard. The results indicate that using this method of detection, it is possible to detect as low as 98 ng/mL (0.49 ng) of HSA and ≦18 ng/mL of IgG in an original sample. The concentrations of albumin and IgG remaining after washing is 440 ng/mL and 140 ng/mL respectively.

The albumin and IgG concentrations in the starting RBCC supernatant are 28±4 mg/mL and 9.7±3.3 mg/mL, therefore the removal level of albumin and IgG according to the method described in Example 1A should be about 4.8 logs.

### EXAMPLE 6

### Preparation of Prion Protein Spiking Material

A. Scrapie Hamster Brain Homogenate Preparation

Scrapie (strain 263K)-infected hamster brains are homogenized in cold phosphate buffered saline (PBS) to a final concentration of 10% (w/v). The homogenate is sonicated (Microsonix Cup Sonicator setting 8-10.3×1 minute) to create a uniform suspension for spiking.

B. Scrapie Hamster Brain Microsomal PrP^{Sc} Preparation

Scrapie (strain 263K)-infected hamster brains are homogenized and sonicated as described above. The preparation is centrifuged at low speed (5,000×g for 10 minutes) to pellet coarse debris. The supernatants are removed and the PrP^{Sc} is pelleted by ultracentrifugation (200,000×g for 30 minutes). The supernatants and liposkins were carefully removed and discarded. The pellets are resuspended in PBS, sonicated to homogeneity and used for spiking.

C. Normal Bovine Brain Homogenate Preparation

Normal bovine brain is homogenized by sonication. PrP^{C} from the homogenate is extracted with 2% Triton X 100. The extracted mixture is partially purified by SP-sepharose chromatography followed by Metal-chelating chromatography. The PrP^{C}-containing fractions are used for spiking.

D. Normal Prion Protein from Human Platelets (huPltPrP^{C})

1. 984 ng/mL Preparation

Platelets from one apheresis unit are washed with HEPES buffer. CaCl₂ and Ca⁺⁺ ionophore is added to induce platelet activation. The activated platelets are ultracentrifuged to pellet out non-soluble proteins at 230,000×g for 1 hour. The supernatant containing the soluble PrP^{C} is used for spiking and comprises PrP^{C} at 984 ng/mL.

2. 5400 ng/ml Preparation

Platelets from six apheresis units are washed with HEPES buffer. CaCl₂ and Ca⁺⁺ ionophore is added to induce platelet activation. Protease inhibitors are added to prevent proteolysis. The activated platelets are ultracentrifuged to pellet out cellular debris and non-soluble proteins at 230,000×g for 1 hour. The supernatant containing the soluble PrP^{C} is concentrated approximately 10 fold using a 10 KDa nominal molecular weight cutoff centrifugal concentrator and the retentate is used for spiking. The spike (45 mL) contains PrP^{C} at 5400 ng/mL.

E. Syrian Hamster Recombinant PrP (Sha rPrP)

The recombinant α and β forms of full-length prion protein are obtained from the TSE Resource Centre, Institute for Animal Health, Compton, Berkshire, UK. Essentially the protein is expressed in E. coli, extracted and purified by IMAC and cation-exchange chromatography under reducing and denaturing conditions. The protein is oxidized and re-folded into its α-form by the CuCl₂ dialysis method of Jackson and colleagues (Jackson, G. S., et al. (1999). Multiple folding pathways for heterologously expressed human prion protein. Biochim Biophys Acta 1431, 1-13). Recombinant PrP β-form was made from rPrP α-form. (Jackson G. S., et al. 1999). Quality control data of the recombinant α and β-forms of the protein are provided by SDS gel analysis, mass spectrometry and circular dichroism. Prior to use in spiking experiments the recombinant proteins are centrifuged at 100 000 g for 1 hour to remove insoluble protein formed on storing or freezing and their concentration determined by UV spectroscopy.

### EXAMPLE 7

### Removal of Prion Protein by Automated Wash Procedure

A. Removal of Scrapie Infected Hamster Brain Homogenate from RBCC

A unit of anti-coagulated RBCC is leukoreduced using a leukofilter e.g., PALL RCXL1 1. A 5% v/v of a 10% preparation of scrapie infected hamster brain homogenate prepared according to Example 6 above (approximately 2 brains or 200 μg of PrP^{SC}), is added to the RBCC and the unit is manually mixed for 1 minute. The RBCC unit is incubated with agitation for one hour at room temperature. The RBCC unit is then washed according to Example 1A.

0.5 mL samples are taken from the washed RBCC unit and lysed with an equal value of 10% sarkosyl. The samples are spun at 130000×g for 30 minutes at room temperature. Endogenous PrP^{C} is soluble and is decanted along with other blood components which remain in the supernatant. The pellets are then washed once with 5% sarkosyl and again ultracentrifuged at 130000×g for 30 minutes at room temperature. The pellets are washed once with PBS to remove remaining sarkosyl and again ultracentrifuged at 130000×g for 30 minutes at room temperature. The final pellets are resuspended in a small volume of 6M Guanidine HCl, 50 mM Tris pH 7.4, sonicated to homogeneity and boiled for 10 minutes. The samples were then assayed directly by time-resolved dissociation-enhanced fluoroimmunoassay (DELFIA^{R}, see, Hemmila Scand. J. Clin. Lab. Invest., 48:389-399, 1988, and MacGregor, et al., Vox Sang. 77: 88-96, 1999) for PrP, or (after denaturation in GdnCl) PrP^{Sc}

The concentration of scrapie hamster brain homogenate is reduced 1.2 to 3.0 log compared to the concentration of scrapie brain homogenate in the unwashed, spiked control. Log removal in the automated and manual wash procedures described herein is determined by comparison of the samples to a standard curve prepared by limiting dilution of the initial spiked WB into non-spiked homologous WB. Alternatively, PrP^{C} levels are quantified by using a standard curve generated from a platelet derived PrP^{C} calibrator. The PrP^{C} calibrator is obtained by detergent treatment (1.0% Triton X100) of washed platelets, followed by centrifugation at 2000×g for 20 min to remove coarse cellular debris. The calibrator is calibrated against a standard curve of known concentration of SHa rPrP and determined to be 709 ng/mL.

### B. Removal of Scrapie Infected Hamster Brain Homogenate from Whole Blood

A 10% hamster scrapie brain homogenate (strain 263K) is prepared as described in Example 6 above. A unit of whole blood (WB) is spiked with a 5% volume of 10% SBH (approximately 3 brains or 300 μg of PrP^{Sc}) and is incubated for 1 hour at 22° C. The unit is then fractionated into an RBCC component (1300×g, 4 min), resuspended in AS-3 solution and leukoreduced through a Pall RCXL1 leukoreduction filter. The leukoreduced unit is washed according to Example 1A above. Samples are taken for analysis prior to leukoreduction, post leukoreduction and following the wash procedure. Endogenous PrP^{C} is removed and PrP^{Sc} is recovered from the samples by the ultracentrifugation procedure and detected as described in Example 7A above.

PrP^{Sc} is reduced by ≧1 to 2.9 logs. 0.1 to 0.8 logs of clearance is attributable to leukoreduction and 1.6 to 2.1 logs of clearance is attributable to the described washing procedure.

C. Removal of Endogenous Human PrP^{C}

A unit of whole blood is fractionated into a RBCC using standard blood banking techniques (1300×g, 4 min). The RBCC is leukofiltered through a PALL RCXL1 leukoreduction filter. The LR-RBCC unit is washed according to the procedure of Example 1A.

Residual PrP^{C} associated with the RBCC is quantified using time-resolved dissociation enhanced fluoroimmunoassay as described in example 7A above. The concentration of endogenous human PrP^{C} is reduced to the level of detection of the assay. The concentration of endogenous human PrP^{C} is reduced about two logs (≧1.8 to 2.0 logs) compared to the concentration of endogenous human PrP^{C} in the unwashed control.

D. Removal of Endogenous Human PrP^{C} and spiked Human Platelet Derived PrP^{C}

Two compatible units of whole blood are fractionated into RBCC's using standard blood banking techniques (1300×g, 4 min). The units are combined and redistributed into identical units. Human platelet derived PrP^{C} (240 ug) prepared as described in Example 6D2 above is spiked into one of the RBCC units and incubated for 1 hour. The second unit receives an equal volume of HEPES buffer. The RBCC units are leukofiltered through a PALL RCXL1 leukoreduction filter. The LR-RBCC units are washed according to the procedure of Example 1A (n=5). Concentrations of endogenous and spiked PrP are determined in cellular and cell free fractions using time-resolved dissociation enhanced fluoroimmunoassay as described in example 7A above. PrP concentrations are determined in cell free samples from a standard curve produced from a platelet derived PrP^{C} calibrator. Reductions for the cellular fractions are based upon serial dilutions of the spiked starting material into the unspiked washed blood.

Analysis of the cellular fractions demonstrates a ≧3.0 log reduction of huPltPrP^{C} by the washing process alone. Furthermore, levels of endogenous PrPc and huPltPrP^{C} are 32.4 ng/mL and 1140 ng/mL respectively before leukoreduction, 16.3 ng/mL and 850 ng/mL respectively before washing and ≦0.14 ng/mL after washing when the acellular component is analyzed. An overall reduction of ≧2.35 logs of endogenous PrP^{C} is achieved, of which 0.3 logs is removed by leukofiltration and washing further reduces endogenous PrP^{C} levels by ≧2 logs. Likewise, an overall reduction of ≧3.9 logs of huPltPrP^{C} is achieved of which 0.13 logs is removed by leukofiltration and washing further reduces endogenous PrP^{C} levels by ≧3.7 logs.

E. Removal of Syrian Hamster recombinant Prp

Two compatible units of whole blood are fractionated into RBCC's using standard blood banking techniques (1300×g, 4 min). The units were combined and redistributed into identical units. The RBCC units are leukofiltered through a PALL RCXL1 leukoreduction filter. E. coli derived recombinant Syrian hamster rPrP (400 ug), prepared as described in Example 6E above, is spiked into one of the RBCC units and incubated for 1 hour. The second unit receives an equal volume of HEPES buffer. The LR-RBCC units are washed according to the procedure of Example 1A. Concentrations of spiked PrP are determined in cellular and cell free fractions using by time-resolved dissociation-enhanced fluoroimmunoassay described in Example 7A above. PrP concentrations are determined in cell free samples by comparison to a standard curve produced from a platelet derived PrP^{C} calibrator. Reductions for the cellular fractions are based upon serial dilutions of the spiked starting material into the unspiked washed blood.

Levels of the alpha form of Sha rPrP in the cell free suspension are 1405 ng/mL before washing and ≦0.12 ng/mL after washing resulting in a ≧4 log reduction of PrP. Analysis of the cellular fractions demonstrates a ≧3.0 log reduction.

### EXAMPLE 8

### Removal of Prion Protein by Manual Wash Procedure

A unit of anti-coagulated RBCC is leukoreduced using a leukofilter e.g., PALL RCXL1. The 25 mL sample with or without a spike is transferred to a 50 mL conical tube. An equal volume of saline/dextrose solution is added to the tube and mixed for two minutes. The material is centrifuged to pellet the red cells (2000×g for 4 minutes). The supernatant is decanted with care not to disturb the red cell layer. Saline/dextrose solution is added to return the contents of the tube to its original volume (25 mL). The process is repeated for a total of 11 washes. The final RBCC pellet is resuspended in AS-3 storage medium.

Where the RBCC contains no spike the time-resolved dissociation-enhanced fluoroimmunoassay described in Example 7A above is used to detect endogenous PrP^{C}. The concentration of endogenous human PrP^{C} is reduced at least two log in the washed sample compared to the concentration of endogenous human PrP^{C} in the unwashed control.

Where bovine PrP^{C} or huPltPrP^{C} prepared as described in Example 6 above is spiked into a 25 mL RBCC sample at 10% v/v, the time-resolved dissociation-enhanced fluoroimmunoassay described in Example 7A above is used to detect PrP^{C}. The concentration of bovine PrP^{C} or huPltPrP^{C} is reduced in the washed sample compared to the concentration of bovine PrP^{C} or huPltPrP^{C} in the unwashed control.

Where scrapie hamster brain homogenate PrP^{Sc} or scrapie hamster brain microsomal PrP^{Sc} prepared as described above in Example 6 is spiked into the 25 mL RBCC sample at 10% v/v, the centrifugal sample preparation steps and the time-resolved dissociation-enhanced fluoroimmunoassay described in Example 7A above is used to detect PrP^{Sc}. The concentration of scrapie hamster brain homogenate PrP^{Sc} or scrapie hamster brain microsomal PrP^{Sc} is reduced compared to the concentrations in the unwashed controls.

### EXAMPLE 9

### Comparison of HSA and IgG Removal to PrP Removal

Units of RBCC are spiked with huPlt PrPc or alpha SHa rPrP as described in Examples 7D and E above and washed according to the method of Example 1A. Samples are removed after each wash cycle and the cell-free fractions are quantitatively analyzed for the presence of HSA and IgG using the method of Example 5B, and PrP is quantified as described in example 7A. Mean initial levels of HSA (n=5), IgG (n=5), huPlt PrPc (n=5), and rPrP (n=2) are 27.9 mg/mL, 9.67 mg/mL, 850 ng/mL, and 1405 ng/mL respectively. The rate of removal of PrP parallels the rate of removal of HSA and IgG during the initial 4 wash cycles after which the levels of PrP fall below the level of sensitivity of the DELFIA assay. Overall log removal of HSA, IgG, huPlt PrPc, and rPrPc throughout the end of the first wash cycle are 1.62, 1.58, 1.60, and 1.52 logs respectively; throughout the end of the second wash cycle are 2.78, 2.69, 2.76, 2.71 logs respectively; throughout the end of the third wash cycle are 3.60, 3.39, 3.61, 3.13 logs respectively, and throughout the end of the fourth wash cycle are 4.17, 3.88, 3.88, and 3.62 logs respectively. HSA and IgG are further removed to levels of 0.44 ug/mL and 0.14 ug/mL in the final washed sample indicating an overall reduction of 4.80 and 4.83 logs respectively.

Quantitation of PrP following the fourth wash is not possible due to levels falling below the level of sensitivity of the assay (0.14 ng/mL).

### EXAMPLE 10

### Assay for Reduction of Blood Mediated Transmission of Spongiform Encephalopathy

10A. Sheep Bioassay for Reduction of Blood Mediated Transmission of Transmissible Spongiform Encephalopathy.

One to five VRQ/VRQ North England Cheviot sheep are fed multiple doses of BSE-infected bovine brain homogenate as described, for example, by Houston, F.; Foster, J. D.; Chong, A.; Hunter, N., and Bostock, C. J. Transmission of BSE by blood transfusion in sheep. Lancet. Sep. 16, 2000; 356(9234):999-1000. Preferably 1-2 gram doses are fed at monthly intervals for the first three months. Two or more units of blood are collected from each sheep, at 10 days, six months, twelve months, eighteen months and at the culling date. At each time point one unit of collected blood is washed according to the procedure of Example 1A or 1B while the remaining unit is maintained at ambient temperature as a control. Reduction of extracellular protein via the procedure of Example 1A or 1B, e.g. reduction of IgG and/or serum albumin and/or cellular prion protein and/or infectious prion protein is monitored as described in any of the Examples 5, 7, or 9 above.

Transfuse a NZ Cheviot (ARQ/ARQ) recipient sheep with the washed RBCC unit from at least one time point (Group A recipient sheep) and a second recipient with the unwashed, whole blood control or a crude, plasma depleted red cell fraction derived from the unwashed control for that time point (Group B recipient sheep).

The recipient sheep are observed for five years for signs of transmissible spongiform encephalopathy as discussed above in the Detailed Description of the Invention and/or assessed for biochemical indicators of the disease. For example, strong evidence for transmission of BSE to transfusion recipients may be obtained by PrP glycotyping (Hope, J., Wood, S. C., Birkett, C. R., Chong, A., Bruce, M. E., Cairns, D., Goldmann, W., Hunter, N., and Bostock, C. J. (1999). Molecular analysis of ovine prion protein identifies similarities between BSE and an experimental isolate of natural scrapie, CH1641. J Gen Virol 80 (Pt 1), 1-4.) or classical lesion profile strain typing methodologies following secondary transmission to panels of inbred laboratory mice.

A significantly lower incidence of disease or biochemical indicators of disease in Group A recipients compared to Group B recipients is scored as a positive for reduction of the risk of transmission of transmissible spongiform encephalopathy via transfusion. A significantly later onset of disease or biochemical indicators of disease in Group A recipients compared to Group B recipients is scored as a positive protraction of time required for onset of disease from potential exposure via blood transfusion.

10B. Mouse Bioassay for Reduction of Blood Mediated Transmission of Transmissible Spongiform Encephalopathy.

Inoculate recipient mice intra-cerebrally with 20 uL of potentially infected RBCC washed according to Example 1A or 1B (Recipient group A) and a second group of recipient mice with 20 uL of the corresponding unwashed whole blood or a crude, plasma-depleted red cell fraction control (Recipient group B). The potentially infected washed RBCC unit and unwashed control is assessed for the concentration of extracellular protein, such as IgG or serum albumin, cellular prion protein and/or pathogenic prion protein as described in Example 5, 7 or 9 above. The recipient mice can be susceptible transgenic lines such as the known lines Tg101L, or TgHu 101L or Tg BoPrP or conventional RIII or C57B1 mice (Bruce, M. E. Will, R. G., Ironside, J. W., McConnell, I., Drummond, D., Suttie, A., McCardle, L., Chree, A., Hope, J., Birkett, C., Cousens, S., Fraser, H., and Bostock, C. J. (1997). Transmissions to mice indicate that ‘new variant’ CJD is caused by the BSE agent. Nature 389, 498-501; Bruce, M. E. (1993). Scrapie strain variation and mutation. Br Med Bull 49, 822-38.)

A significantly lower incidence of disease or biochemical indicators of disease in Group A recipients compared to Group B recipients is scored as a positive for reduction of the risk of transmission of transmissible spongiform encephalopathy via transfusion. A significantly later onset of disease or biochemical indicators of disease in Group A recipients compared to Group B recipients is scored as a positive protraction of time required for onset of disease from potential exposure via blood transfusion.

### Example 11

### Reduction of Transmission of Transmissible Spongiform Encephalopathies via Transfusion

A human RBCC unit is washed according to Example 1A or 1B above. The human RBCC is assayed for concentration of extracellular protein, such as IgG or serum albumin, prion protein and/or pathogenic prion protein as described in Example 5, 7 or 9 above. RBCC comprising an extracellular protein concentration that correlates to that in a RBCC unit scored positive in Example 10 above is transfused to a recipient.

### OTHER EMBODIMENTS

From the above description, one skilled in the art can easily ascertain the essential characteristics of the present invention. Without departing from the spirit and scope thereof, one of ordinary skill in the art can make various changes and modifications of the invention to adapt it to various uses and conditions. Other embodiments are also within the claims.

What is claimed is:

1. A method for reducing the concentration of an analyte in a blood cell suspension, the method comprising:
(i) providing a starting blood cell suspension in a volume greater than 50 mL, the blood cell suspension comprising blood cells and extracellular fluid; and
(ii) washing the starting blood cell suspension with a wash solution under conditions sufficient to lower the concentration of the analyte at least 10³-fold relative to the analyte concentration in the starting blood cell suspension, wherein the blood cells of the blood cell suspension retain viability after a storage period of greater than 21 days at 4° C. in a storage solution.

2. The method of claim 1, wherein the washing comprises
(i) centrifuging the starting blood cell composition to form a pelleted cell fraction and a supernatant;
(ii) removing the supernatant from the pelleted cell fraction;
(iii) adding washing solution to the pelleted cell fraction; and.
(iv) resuspending the pelleted cell fraction in the washing solution to form a resuspended cell suspension;
(v) optionally repeating steps (i)-(iv); and
(vi) resuspending the pelleted cell fraction in a storage solution.

3. The method of claim 2, wherein the analyte is a small molecule.

4. The method of claim 3, wherein the small molecule is an ethyleneimine oligomer, phenothiazine derivative, acridine derivative, psoralen derivative or riboflavin.

5. The method of claim 3, wherein the small molecule is a therapeutic agent.

6. The method of claim 2, wherein the analyte is a protein.

7. The method of claim 6, wherein the protein is a prion protein.

8. The method of claim 7, wherein the prion protein is a pathogenic protein.

9. The method of claim 2, wherein the analyte is a cell.

10. The method of claim 9, wherein the cell is a leukocyte.

11. The method of claim 10, wherein the method further comprises treating the starting blood cell suspension with an anti-pathogenic agent.

12. The method of claim 11, wherein the anti-pathogenic agent is an ethyleneimine oligomer, phenothiazine derivative, acridine derivative, psoralen derivative or riboflavin.
